(19) United States
(12) Patent Application Publication
(10) Pub. No.: US 2026/0183564 A1
(43) Pub. Date: Jul. 2, 2026

(54) WIRELESS MAGNETOELECTRIC LIGHT SOURCE

(71) Applicant: UNIVERSITÄT ZU KÖLN, Köln (DE)

(72) Inventors: Malte C. GATHER, Köln (DE); Julian F. BUTSCHER, Köln (DE)

(21) Appl. No.: 18/858,860

(22) PCT Filed: Apr. 20, 2023
(86) PCT No.: PCT/EP2023/060336

§ 371 Date: Oct. 22, 2024

(30) Foreign Application Priority Data
Apr. 22, 2022 (DE) 10 2022 109 762.5

(51) Int. Cl. A61N 5/06

(52) U.S. Cl. CPC A61N 5/0622; A61N 5/0601; A61N 2005/0652; A61N 2005/0653; A61N 2005/0664

(57) ABSTRACT

The disclosure relates to a system for providing an optical signal including an energy converter and a light source. The energy converter includes at least two materials and is designed as a magnetoelectric converter for the purpose of generating electrical energy on the basis of a magnetic alternating field. The light source is arranged on the energy converter and electrically conductively contacts a first surface of the energy converter functioning as an electrode. The light source is not electrically contacted with the energy converter via a cable connection.

### INTRODUCTION

The disclosure relates to a system for providing an optical signal.

The disclosure furthermore relates to the use of the above system for optical stimulation of a nerve cell.

The disclosure furthermore relates to a method for producing the above system.

Optogenetics is a biological technology which, by way of genetic modification of neuron cells, enables their stimulation with the aid of light, due to which the selective research of biological neural networks is possible. For example, knowledge about neurodegenerative diseases such as Parkinson's and/or Alzheimer's can thus be collected.

While the biotechnological tools of optogenetics have improved rapidly in recent years, the development of adequate light sources for providing the optical signal for the cell still lags behind. Since an implantation of the light sources into the biological system to be studied can be intended upon use, the light source is to be wirelessly operable to minimize foreign body contact and moreover as small as possible in order to keep the damage due to the implantation as minor as possible

The extremely compact low-power voltage transformer for low-frequency pulsed and alternating voltages is already known from DE 100 25 028 A1, which, with relatively high efficiency and transmission factors up to 10,000-fold, ensures a reliable galvanic separation between input and output sides with the least insulation expenditure in the tightest space.

WO 2020/206 332 A1 describes novel devices, systems, and methods which use magnetoelectric nerve stimulators with tunable amplitude and waveform. Specific embodiments comprise a magnetoelectric film, a magnetic field generator, and an electric circuit which is coupled to the magnetoelectric film, wherein in certain embodiments the electric circuit comprises components configured so as to modify an electrical output signal generated by the magnetoelectric film.

The ignition pulse generator from WO 01/91 200 A1 is a voltage transformer for generating high-voltage pulses up to 30 kV. The ignition pulse generator is composed of a magnetomechanical transducer, consisting of the magnetostrictive actuator core and the coil surrounding it having a flux guide plate, an insulator, and an electromechanical transducer, which is embodied as a piezoelectric ignition element. The transformation of the voltage takes place due to the simultaneous usage of the magnetostrictive effect and the direct piezoelectric effect. The ignition pulse generator is used for igniting technical gases and for electrically igniting pyrotechnic primers.

A device-called a harvester- and a method for collecting energy from the environment and/or other external sources and its conversion into useful electrical energy are known from U.S. Pat. No. 7,808,236 B1. The harvester does not contain permanent magnets or another local field source, but rather is based on the Earth's magnetic field or another source of a magnetic field located outside the sensor device. One advantage of this novel harvester is that it can be constructed smaller and lighter than energy harvesters which contain a magnet and/or an inertial mass.

WO 2010/097 407 A1 relates to a light source which comprises a piezoelectric transformer and one or more devices based on semiconductors, which display electroluminescence and are electrically connected thereto. The piezoelectric transformer is configured so that an inherent electrical property defines a predetermined upper limit for an output current which is available from an output terminal of the piezoelectric transformer.

A device, in particular a microsystem, is described in DE 10 2006 040 731 A1, which comprises an apparatus for energy conversion. The apparatus for energy conversion has a piezoelectric, mechanically oscillating membrane structure for converting mechanical energy into electrical energy and/or vice versa, wherein the membrane structure is arranged encapsulated in an environment which has a predetermined pressure that is in particular lower than an isostatic pressure.

US 2016/0 303 402 A1 comprises methods and devices for modulation of the activity or the activities of living cells, such as cells which occur in humans, animals, plants, insects, microorganisms, and other organisms or originate therefrom. The methods comprise the application of ultrasound, such as ultrasound having low intensity and low frequency, to living cells in order to influence the cells and modulate the activities of the cells.

### SUMMARY

Preceding therefrom, it is an object of the disclosure, per an embodiment, to provide a light source which is wirelessly operable and can be reduced in size particularly easily.

According to the disclosure, per an embodiment, a system for providing an optical signal comprising an energy converter and a light source is provided, wherein the energy converter comprises at least two materials and is designed as a magnetoelectric converter for the purpose of generating electrical energy on the basis of a magnetic alternating field, wherein the light source is arranged on the energy converter and electrically conductively contacts a first surface of the energy converter functioning as an electrode and wherein the light source is not electrically contacted via a cable connection to the energy converter.

One aspect of the disclosure, per an embodiment, is that the energy supply of the light source is provided via the energy converter, wherein the energy converter is designed as a magnetoelectric converter for the purpose of generating electrical energy on the basis of a magnetic alternating field. This enables the light source to be operated wirelessly.

A further aspect of the disclosure, per an embodiment, is that the light source is arranged on the energy converter. It is provided here that an electrically conductive connection is present between the light source and the first surface of the energy converter functioning as an electrode. The light source can be arranged touching the first surface and can contact it flatly. Alternatively, the light source can be arranged on the energy converter but not contact the first surface flatly, but rather can only be electrically conductively connected to the first surface via one or more layers. In both cases, the energy converter is used as the substrate for the light source. In this way, it is possible for cable connections to be eliminated as the electrical connection between the energy converter and the light source. Accordingly, the system can be embodied particularly small and can moreover be miniaturized particularly well.

Moreover, it is provided in this context that the light source is not electrically contacted with the energy converter via a cable connection. The system therefore does not have a cable connection as an electrical conductor between the light source and the energy converter. In this way, the system is particularly small and robust. This makes the system particularly suitable to be implantable into a biological system to be studied.

In conjunction with the energy converter, it is provided according to one refinement, per an embodiment, that one of the at least two materials is embodied as a piezoelectric material and the other of the at least two materials is embodied as a magnetostrictive material.

A piezoelectric material is a material in which piezoelectricity, also called the piezoelectric effect, occurs-that is to say the occurrence of an electrical voltage when the material is elastically deformed. A magnetostrictive material is a material in which a deformation occurs as a result of an applied magnetic field. The energy converter is thus based on the composite magnetoelectric effect, in which the properties of magnetostrictive and piezoelectric materials are connected to one another by mechanical coupling. The magnetostrictive material deforms in the magnetic field, which results in an elastic deformation of the piezoelectric material, by which an electrical voltage is generated. The design of the energy converter in this form has the advantage, per an embodiment, that in spite of the small size of the energy converter for generating electrical energy, a low-frequency magnetic alternating field can be used, which is hardly attenuated by biological tissue. Accordingly, the light source can also be operated wirelessly in a state surrounded by biological tissue without significant energy losses. A further advantage, per an embodiment, is that the energy converter can be miniaturized particularly well.

The piezoelectric material is particularly selected from the group comprising piezoelectric ceramics, piezoelectric crystals, and/or piezoelectric plastics and in particular lead-zirconate-titanate (PZT), lead-magnesium-niobate-lead-titanate (PMN-PT), and/or polyvinylidene fluoride (PVDF). These materials have thus proven to be particularly suitable for the energy converter.

The magnetostrictive material is particularly selected from the group comprising metallic glasses and/or amorphous metals and in particular iron-silicon-borite (FeSiB), iron-cobalt-silicon borite (FeCoSiB), and/or nickel. These materials have thus proven to be particularly suitable for the energy converter. Further, the piezoelectric material is PZT and the magnetostrictive material is FeSiB.

With respect to the light source, it is provided that the light source comprises multiple organic light-emitting diodes. An organic light-emitting diode (OLED) is a luminous thin film component made of organic semiconducting materials. The OLED moreover comprises two electrically conductive light source electrodes. OLEDs have the advantage, per an embodiment, that they can be produced cost-effectively in thin-film technology and can be applied particularly easily to the energy converter in this way. In other words, a plurality of OLEDs, that is to say at least two OLEDs, are applied to the energy converter. Furthermore, it is provided that the OLEDs are designed to generate an optical signal in the visible and/or infrared range, preferably between 380 nm and 1000 nm. This makes the optical stimulation of nerve cells particularly simple.

According to one refinement of the disclosure, per an embodiment, it is provided that the light source comprises two OLEDs connected antiparallel. Connected antiparallel means that in a parallel circuit of the OLEDs, in which two current paths are formed, one of the two OLEDs is connected in the blocking direction and the other of the two OLEDs is connected in the forward direction. By means of this electrical interconnection of two OLEDs, in spite of the capacitive properties of the energy converter, which does not behave like an ideal alternating voltage source but rather basically like a periodically charging and discharging capacitor, an optical signal can be generated. This furthermore has the advantage, per an embodiment, that during use of the system, an optical signal is generated both during the positive and during the negative sinusoidal voltage curve of the energy converter. Due to the operating frequencies of the energy converter which are typically used, the light source moreover appears to be continuously luminous to the human eye. The two OLEDs can be designed identically, thus generate an optical signal having equal frequency. Alternatively, the two OLEDs can be designed differently and can each generate an optical signal at frequencies different from one another.

In principle, the two OLEDs can be arranged adjacent to one another. According to another refinement of the disclosure, per an embodiment, however, it is provided that the light source comprises two OLEDs stacked one on top of the other. This is a particularly easily reproducible and space-saving option for attaching the light source to the energy converter. In particular, stacked one on top of the other means that one of the two OLEDs is applied directly to the energy converter and in particular directly on the first surface of the energy converter functioning as the electrode and the other of the two OLEDs is applied to the first OLED and in this way a stacked arrangement is achieved. This has the advantage, per an embodiment, that the two OLEDs can be deposited in succession by means of coating technologies during the production, so that the production of the system is particularly simple and robust.

Furthermore, it can also be provided that the light source comprises a plurality of OLEDs and in particular comprises more than two OLEDs. It is provided that at least two of the plurality of OLEDs are connected antiparallel to one another. The orientation of the circuit of the further OLEDs can be freely selected. Furthermore, it is provided that the plurality of OLEDs are stacked one on top of another in one stack or in multiple stacks. Arbitrary color mixtures such as white light as well as a local separation of the light sources can be achieved using a plurality of OLEDs.

As already mentioned, the light source electrically conductively contacts the first surface of the energy converter functioning as an electrode. It can be provided here that further layers of the system are arranged between the light source and the first surface of the energy converter. Alternatively, it is provided that the light source is applied directly to the first surface of the energy converter and touches it and in this way an electrically conductive connection is present between the first surface and the light source.

Furthermore, the energy converter comprises two surfaces opposite to one another, which both function as electrodes. Furthermore, the energy converter is designed as a magnetoelectric converter for the purpose of generating an electrical alternating voltage via its two opposing surfaces on the basis of the magnetic alternating field.

Moreover, the first and/or the second of the two surfaces of the energy converter are partially insulated. In other words, it is thus provided that the light source is applied to the first of the two partially insulated surfaces of the energy converter acting as the electrode and preferably contacts the first surface by touching in such a way that an electrically conductive connection is present between the first surface of the energy converter functioning as an electrode and the light source.

Furthermore, the light source comprises two light source electrodes, wherein one of the two light source electrodes is contacted with the first surface of the energy converter functioning as an electrode. In this context, it is moreover provided according to a further refinement, per an embodiment, that the light source is electrically conductively contacted with the second surface of the energy converter functioning as an electrode via a conductive layer and an insulating layer electrically insulates the conductive layer from the first surface. In other words, the second light source electrode is thus electrically insulated by the insulating layer from the first surface of the energy converter. The partially insulated energy converter is thus used as a substrate for the light source which is applied over the first of the two surfaces of the energy converter functioning as an electrode and over the partial insulation of this surface. The second light source electrode is connected via the conductive layer-preferably a conductive thin-film connection-to the opposite second surface of the energy converter functioning as an electrode, so that the electric circuit is closed.

With respect to the insulating layer, it is furthermore provided that it electrically insulates all surfaces of the energy converter except for a part in each case of the first and second surface of the energy converter. The light source is contacted on the uninsulated part. The insulating layer is used for electrically separating the two light source electrodes and avoiding an electrical short circuit between the two first and second surfaces of the energy converter functioning as electrodes. The insulating layer comprises an insulating material selected from the group comprising organic polymers or metallic oxides and in particular parylene C and aluminum (III) oxide. These materials have thus proven to be particularly suitable for the electrical insulation.

With regard to the energy converter, the energy converter can be embodied differently. For example, the first material can be present in the form of small particles, preferably nanoparticles in the first material, wherein the particles can be homogeneously distributed, for example. Alternatively, the first material can permeate the second material in the form of columns or other structures. However, it is provided according to a further refinement of the disclosure, per an embodiment, that the energy converter has a layered structure having at least two layers, wherein the first material and the second material are each formed as a layer of the energy converter and wherein the light source is applied to the first of the two layers and electrically conductively contacts it. A layered structure is particularly easy to produce.

In principle, it can be provided that the first layer is embodied as a layer having piezoelectric material. Alternatively, it can be provided that the first layer is embodied as a layer having magnetostrictive material. In a further refinement of the disclosure, per an embodiment, however, it is provided that the first layer is embodied as a layer having magnetostrictive material. In other words, the light source thus preferably contacts the layer having magnetostrictive material of the energy converter or in still other words the light source is preferably applied to the layer having magnetostrictive material. This embodiment has the advantage, per an embodiment, over the application of the light source to the layer having piezoelectric material that during the production, the layer having magnetostrictive material is smoother on the surface than the layer having piezoelectric material, so that the surface of the layer having magnetostrictive material does not have to be polished before application of the light source. The production of the system is accordingly simplified.

According to a further refinement of the disclosure, per an embodiment, it is provided that a connecting layer is arranged between the first layer and the second layer. In other words, it is provided that the first and the second layer do not touch directly but rather the connecting layer is present therebetween. The connecting layer ensures the mechanical coupling of the first and second layers. The connecting layer is an epoxy layer. In this way, the energy converter can be produced in a simple manner by connecting the first and second layers by means of the epoxy layer. Furthermore, it is provided that the connecting layer presses directly touching on the layer having magnetostrictive material and/or on the layer having piezoelectric material.

Furthermore, it is provided in conjunction with the layered structure of the energy converter that the layer having magnetostrictive material comprises a homogeneous layer comprising one or more magnetostrictive materials. A layer thickness of the homogeneous layer comprising one or more magnetostrictive materials is preferably between 1 μm and 100 μm. Furthermore, it is provided that the light source is applied to the homogeneous layer comprising one or more magnetostrictive materials. Furthermore, it is likewise provided that the layer having magnetostrictive material exclusively consists of the homogeneous layer comprising one or more magnetostrictive materials.

Moreover, it is furthermore provided that the layer having piezoelectric material comprises a homogeneous layer comprising one or more piezoelectric materials. Furthermore, a layer thickness of the homogeneous layer comprising one or more piezoelectric materials is preferably between 1 μm and 500 μm. In principle, the layer having piezoelectric material can also exclusively consist of the homogeneous layer comprising one or more piezoelectric materials. However, since some piezoelectric materials are not intrinsically conductive, it is provided that the homogeneous layer comprising one or more piezoelectric materials is provided with a conductive layer on its upper side and on its lower side, however. In other words, the layer having piezoelectric material also comprises two conductive layers, in addition to the homogeneous layer comprising one or more piezoelectric materials. The conductive layers comprise or consist of metal and particularly of nickel.

According to a further refinement of the disclosure, per an embodiment, a system is provided in which the light source and the energy converter are integrated in an encapsulation. In other words, the energy converter and the light source applied to the energy converter are thus encapsulated and/or integrated in the encapsulation. The system can be protected in a simple manner in this way from harmful environmental influences such as oxygen and/or moisture. Furthermore, it is provided that the encapsulation is made of a biocompatible material. The encapsulation is particularly optically transparent, thus has an optical transmission of at least 80% for the optical signal in the visible range. The encapsulation comprises parylene C combined with alternating layers made of aluminum oxide and zirconium oxide.

According to a further refinement of the disclosure, per an embodiment, it is provided that the system comprises an energy source, wherein the light source and the energy source and/or the energy converter and the energy source are not connected to one another via an electrical connection. In other words, a cable connection between the energy source and the energy converter and/or the light source is thus omitted. The light source is thus operated wirelessly during use of the system.

In this context, it is provided according to a further refinement of the disclosure, per an embodiment, that the energy source is designed as a coil and/or is designed to generate a magnetic alternating field. The energy transmission thus takes place via the magnetic alternating field from the energy source to the energy converter. The energy source is designed to generate a magnetic alternating field at a frequency between 50 KHz and 2 MHZ. This frequency range has the advantage, per an embodiment, that the magnetic alternating field is only slightly absorbed by biological tissue and in this manner there are hardly any energy losses. Furthermore, it is provided that the energy source is designed to generate a magnetic alternating field at a frequency which is resonant with an oscillation frequency of the energy converter. In other words, the energy source and the energy converter are preferably tuned to one another.

Furthermore, it is provided that the energy converter and the light source are designed for optical stimulation of nerve cells. This means, per an embodiment, that the light source and the energy converter together preferably comprise dimensions below 11 mm in the longest external dimension. A longest lateral dimension of the device comprising the energy converter and the light source is particularly less than 1 mm, more preferably less than 800 μm, and particularly less than 500 μm.

The disclosure, per an embodiment, furthermore relates to the use of the above-described system for the optical stimulation of a nerve cell. It can be provided here that the system is used to provide an optical signal in vitro and/or ex vivo, or in other words that the nerve cell is optically stimulated outside a living biological organism by means of the system. Alternatively, the system can be used in vivo.

The disclosure, per an embodiment, furthermore relates to a method for producing a system for providing an optical signal, and preferably for producing the above-described system, comprising the following steps
a) providing an energy converter, wherein the energy converter comprises at least two materials and is designed as a magnetoelectric converter for the purpose of generating electrical energy on the basis of a magnetic alternating field,
b) applying a light source to the energy converter by vacuum-based coating technology in such a way that the light source electrically conductively contacts a first surface of the energy converter functioning as an electrode and in such a way that the light source is not electrically contacted with the energy converter via a cable connection.

In other words, the energy converter is thus used as a substrate for the light source, which is applied directly to the energy converter. The light source is deposited by means of vacuum-based coating technology on the first surface of the energy converter functioning as an electrode, in such a way that the light source rests by touching on the first surface. A miniaturized system can be produced particularly easily in this way. However, it can also be provided that the first surface of the energy converter is partially coated using an insulation and the light source is applied to the insulation such that the insulation is between the light source and the first surface and at the same time the light source electrically conductively contacts the first surface at an edge, for example.

With regard to the above-described layered structure of the energy converter, it is provided that the light source is deposited on the first layer and particularly on the layer having magnetostrictive material of the energy converter. In other words, the first surface functioning as an electrode is thus preferably a layer having magnetostrictive material of the energy converter.

Furthermore, it is provided that step a) providing the energy converter comprises the following steps:
providing two layers, wherein one of the two layers is designed as a layer having piezoelectric material and another of the two layers is designed as a layer having magnetostrictive material, and
connecting the two layers by means of a connecting layer preferably made of epoxy, wherein the connecting layer is applied by means of rotation coating to one of the two layers.

In this way, the magnetoelectric energy converter can be provided in a simple manner, which is then furthermore used as the substrate for applying the light source.

Furthermore, step b) applying the light source to the energy converter by vacuum-based coating technology such that the light source electrically conductively contacts the first surface of the energy converter functioning as an electrode preferably comprises vapor depositing individual layers of at least two OLEDs using shading masks. The two OLEDs are particularly vapor-deposited such that they are stacked one on top of the other.

Furthermore, step b) comprises the following steps:
depositing an insulating layer on the energy converter such that the insulating layer directly touches the first surface and/or layer,
depositing a layer of electrically conductive material on the insulating layer by means of atomic layer deposition,
partially removing the layer of electrically conductive material and the insulating layer by means of chemical and/or physical methods such that a part of the first surface and/or layer is exposed, and
applying the light source to the exposed part of the first surface and/or layer such that the light source electrically conductively contacts the first surface and/or layer.

Alternatively, it can be provided in the last step that the light source is applied to the insulating layer such that the light source electrically conductively contacts the exposed part of the first surface and/or layer.

Moreover, the layer of electrically conductive material is particularly deposited such that it electrically contacts the second layer of the energy converter functioning as a further electrode and the light source is applied to the exposed part of the first layer such that the light source electrically conductively contacts the first layer and electrically conductively contacts the second layer of the energy converter via the layer of electrically conductive material

Furthermore, the method moreover comprises the following step
encapsulating the energy converter and the light source in an encapsulation such that the light source and the energy converter are integrated in the encapsulation. The encapsulation is applied by means of gas phase deposition to the energy converter and the light source.

### BRIEF DESCRIPTION OF THE FIGURES

The disclosure is explained by way of example hereinafter with reference to the appended drawings on the basis of exemplary embodiments, wherein the features described hereinafter can represent an aspect of the disclosure both individually and in combination. In the figures:

FIG. 1 shows a schematic sectional view of a system for providing an optical signal according to an embodiment of the disclosure,

FIG. 2 shows a schematic view of an electrical circuit diagram of the system from FIG. 1, and

FIG. 3 shows a schematic representation of a further embodiment of a system for providing an optical signal according to a further embodiment of the disclosure.

### DETAILED DESCRIPTION

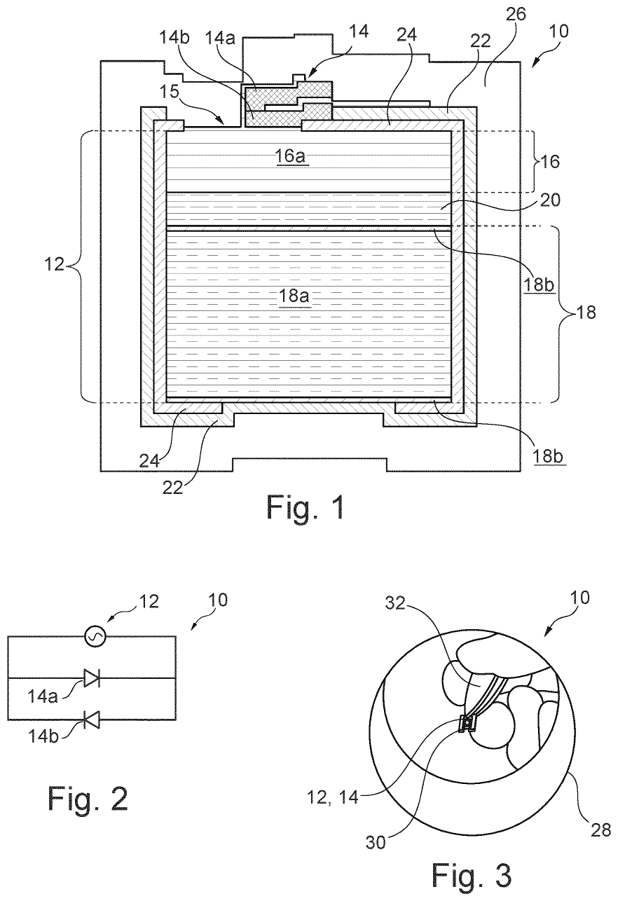

FIG. 1 shows a schematic sectional view of a system 10 for providing an optical signal. The system 10 comprises an energy converter 12 and a light source 14, wherein the energy converter 12 comprises at least two materials 16a, 16b and is designed as a magnetoelectric converter for the purpose of generating electrical energy on the basis of a magnetic alternating field. The light source 14 is arranged on the energy converter 12 and electrically conductively contacts a surface 15 of the energy converter 12 functioning as a first electrode.

Moreover, the energy converter 12 has a layered structure having at least two layers 16, 18 in the present case. As can be seen in FIG. 1 in the present case, the first layer 16 of the energy converter 12 is designed as a homogeneous layer comprising the magnetostrictive material 16a, wherein Metglas 2605SA1 (FeSiB alloy) 16a is used as the magnetostrictive material. The second layer 18 of the energy converter 12 is designed in the present case as a layer 18 having piezoelectric material 18a. The second layer 18 comprises the homogeneous layer comprising the piezoelectric material 18a lead-zirconate-titanate 18a, and moreover two layers of nickel 18b, which are each applied directly on one side of the lead-zirconate-titanate 18a layer. The first layer 16 and the second layer 18 of the energy converter 12 are connected to one another via a connecting layer 20—in the present case made of epoxy.

The light source 14 is applied in the present case to the first surface 15 of the energy converter 12, which also means in the present case that the light source 14 is applied to the first layer 16 of the energy converter 12 and electrically conductively contacts the first layer 16. As can moreover be seen in FIG. 1, the system 10 has a third layer 22 and an insulating layer 24. The light source 14 is electrically conductively contacted via the third layer 22 with the second layer 18 of the energy converter 12, wherein the insulating layer 24 electrically insulates the third layer 22 from the first layer 16.

Furthermore, it can be seen in FIG. 1 that the light source 14 comprises two OLEDs 14a, 14b stacked one on top of the other. Moreover, the light source 14 and the energy converter 12 are integrated in an encapsulation 26—in the present case made of parylene C.

FIG. 2 shows a schematic view of an electrical circuit diagram of the system 10 from FIG. 1. As can be seen therein, the two OLEDs 14a, 14b of the light source 14 are connected antiparallel to one another, so that each of the two OLEDs 14a, 14b has its own circuit, and one of the two OLEDs 14a, 14b is connected in the blocking direction and the other of the two OLEDs 14a, 14b is connected in the forward direction.

FIG. 3 shows a schematic representation of a further embodiment of a system 10 for providing an optical signal according to a further embodiment of the disclosure. In this embodiment, the system 10 also comprises, in addition to the energy converter 12 and the light source 14, an energy source 28 designed as a coil 28. The energy converter 12 and the light source 14 are furthermore also applied to a carrier 30, so that this can be held in the coil 28 by means of tweezers 32 to generate the optical signal in spite of the small dimensions of the energy converter 12 and the light source 14 of 11 mm×3 mm×0.16 mm taken together.

As used herein, the terms “general,” “generally,” and “approximately” are intended to account for the inherent degree of variance and imprecision that is often attributed to, and often accompanies, any design and manufacturing process, including engineering tolerances, and without deviation from the relevant functionality and intended outcome, such that mathematical precision and exactitude is not implied and, in some instances, is not possible.

All the features and advantages, including structural details, spatial arrangements and method steps, which follow from the claims, the description and the drawing can be fundamental to the invention both on their own and in different combinations. It is to be understood that the foregoing is a description of one or more preferred exemplary embodiments of the invention. The invention is not limited to the particular embodiment(s) disclosed herein, but rather is defined solely by the claims below. Furthermore, the statements contained in the foregoing description relate to particular embodiments and are not to be construed as limitations on the scope of the invention or on the definition of terms used in the claims, except where a term or phrase is expressly defined above. Various other embodiments and various changes and modifications to the disclosed embodiment(s) will become apparent to those skilled in the art. All such other embodiments, changes, and modifications are intended to come within the scope of the appended claims.

As used in this specification and claims, the terms “for example,” “for instance,” “such as,” and “like,” and the verbs “comprising,” “having,” “including,” and their other verb forms, when used in conjunction with a listing of one or more components or other items, are each to be construed as open-ended, meaning that the listing is not to be considered as excluding other, additional components or items. Other terms are to be construed using their broadest reasonable meaning unless they are used in a context that requires a different interpretation.

### LIST OF REFERENCE NUMERALS

10 system
12 energy converter
14 light source
14a first OLED
14b second OLED
15 first surface
16 first layer
16a magnetostrictive material
18 second layer
18a piezoelectric material
18k nickel
20 connecting layer
22 third layer, conductive layer
24 insulating layer
26 encapsulation
28 coil
30 carrier
32 tweezers

1. A system for providing an optical signal comprising an energy converter and a light source,
wherein the energy converter comprises at least two materials and is designed as a magnetoelectric converter for the purpose of generating electrical energy on the basis of a magnetic alternating field,
wherein the light source is arranged on the energy converter and electrically conductively contacts a first surface of the energy converter functioning as an electrode, and
wherein the light source is not electrically contacted with the energy converter via a cable connection.

2. The system as claimed in claim 1, wherein one of the at least two materials is designed as a piezoelectric material and the other of the at least two materials is designed as a magnetostrictive material.

3. The system as claimed in claim 1, wherein the light source comprises two OLEDs connected antiparallel.

4. The system as claimed in claim 1, wherein the light source comprises two OLEDs stacked one on top of the other.

5. The system as claimed in claim 1, wherein the light source is electrically conductively contacted via a conductive layer with a second surface of the energy converter functioning as an electrode and an insulating layer electrically insulates the conductive layer from the first surface.

6. The system as claimed in claim 1, wherein the energy converter has a layered structure having at least two layers, wherein a first material and a second material of the at least two materials are each formed as a layer of the energy converter and wherein the light source is applied to the first of the two layers and electrically conductively contacts it.

7. The system as claimed in claim 6, wherein the first layer is designed as a layer having magnetostrictive material.

8. The system as claimed in claim 1, wherein a connecting layer is arranged between a first layer and a second layer of the at least two materials.

9. The system as claimed in claim 1, wherein the light source and the energy converter are integrated in an encapsulation.

10. The system as claimed in claim 1, comprising an energy source, wherein the light source and the energy source and/or the energy converter and the energy source are not connected to one another via an electrical connection.

11. The system as claimed in claim 10, wherein the energy source is designed as a coil and/or is designed to generate a magnetic alternating field.

12. A use of a system as claimed in claim 1 for optical stimulation of a nerve cell.

13. A method for producing a system for providing an optical signal, comprising the following steps:
a) providing an energy converter, wherein the energy converter comprises at least two materials and is designed as a magnetoelectric converter for the purpose of generating electrical energy on the basis of a magnetic alternating field, and
b) applying a light source to the energy converter by vacuum-based coating technologies such that the light source electrically conductively contacts a first surface of the energy converter functioning as an electrode and such that the light source is not electrically contacted with the energy converter via a cable connection.
